(19) United States
(12) Patent Application Publication
(10) Pub. No.: US 2008/0188866 A1
(43) Pub. Date: Aug. 7, 2008

(54) APPARATUS AND METHODS FOR REMOVING RELATIVELY LARGE AND SMALL STONES FROM A BODY PASSAGE

(75) Inventors: John A. Karpiel, Winston-Salem, NC (US); Wenfeng Lu, Pfafftown, NC (US); Brian K. Rucker, King, NC (US)

Correspondence Address: BRINKS HOFER GILSON & LIONE/CHICAGO/COOK, PO BOX 10395, CHICAGO, IL, 60610, US

(73) Assignee: Wilson-Cook Medical Inc., Winston-Salem, NC (US)

(21) Appl. No.: 12/024,503

(22) Filed: Feb. 1, 2008

Related U.S. Application Data
(60) Provisional application No. 60/899,478, filed on Feb. 5, 2007.

(51) Int. Cl. A61B 17/22

(52) U.S. Cl. 606/127

(57) ABSTRACT

The present invention provides apparatus and methods suitable for removing at least one stone in a body passage. The apparatus comprises a balloon catheter having at least one working lumen adapted to receive an extraction basket. In use, the extraction basket may be used to capture and remove relatively large stones, and/or crush relatively large stones into smaller stone fragments. The balloon may then be inflated and maneuvered to engage the stone fragments and urge at least one of the stone fragments out of the body passage.

### PRIORITY CLAIM

This invention claims the benefit of priority of U.S. Provisional Application Ser. No. 60/899,478, entitled “Apparatus and Methods for Removing Relatively Large and Small Stones from a Body Passage,” filed Feb. 5, 2007, the disclosure of which is hereby incorporated by reference in its entirety.

### TECHNICAL FIELD

The present invention relates generally to apparatus and methods for removing stones from a body passage, and in particular, to a balloon catheter adapted to receive an extraction basket to facilitate removal of relatively large and small stones.

### BACKGROUND INFORMATION

It is common for various calculi, or “stones,” to form within body passages, such as kidney stones in the ureter or kidneys, and gallstones in bile ducts or the gallbladder. Some stones may be harmless and may pass through the body naturally, for example, gallstones passing through the duodenum and kidney stones through the urethra. However, many other stones may become trapped and may cause serious medical problems, such as abdominal pain, fever, nausea, jaundice, and so forth. Fast and effective removal of such stones may become necessary.

In order to remove relatively large or trapped stones, it may be necessary to disintegrate a stone into smaller fragments. Several procedures are known for disintegrating the stone and subsequent removal of the smaller stone fragments.

Some common procedures for disintegrating gallstones and kidney stones include electrohydraulic lithotripsy, which uses a small probe to break up stones using shock waves generated by electricity. Similarly, laser lithotripsy may be used to break up stones by directing a controlled laser beam onto the stone surface. Another treatment option is ultrasonic lithotripsy, which uses high frequency sound waves. Alternatively, extracorporeal shock wave lithotripsy (“ESWL”) may be used, which utilizes focused impulses projected from outside the body to disintegrate larger stones. Still other disintegration techniques may be used.

Once larger stones are reduced to smaller sizes using any of the above techniques, the smaller stone fragments may pass naturally through the body, or a stone removal device may be used to extract the stone fragments. Typical extraction devices comprise extraction baskets or extraction balloon catheters. An extraction basket may comprise a plurality of wires that deploy in a radially outward direction and are designed to trap the floating stones. An extraction basket may be especially useful for catching and/or crushing larger stones. However, one limitation associated with extraction baskets is that smaller stone fragments may escape between the basket wires.

As an alternative to an extraction basket, a balloon catheter may be inserted through a working lumen of an endoscope to help remove stone fragments. In an exemplary procedure, the balloon is positioned adjacent to and upstream from the stone, inflated, and then moved in a downstream direction to sweep the stone out of the bile duct and into the duodenum.

However, while separate lithotripsy devices, extraction baskets and extraction balloons are known, the use of such separate devices generally requires the removal of one device prior to the introduction and advancement of a subsequent device. There is no comprehensive system that provides each of these features in one easy-to-use system, thereby facilitating extraction of relatively large and small stones, and reducing the operation time during a stone removal procedure.

### SUMMARY

The present invention provides apparatus and methods suitable for removing at least one stone in a body passage. The apparatus comprises a balloon catheter having a working lumen adapted to receive an extraction basket. In use, the extraction basket may be used to capture and remove relatively large stones, and/or crush relatively large stones into smaller stone fragments. The balloon may then be inflated to engage the stone fragments and urge at least one of the stone fragments and sludge out of the body passage. For example, if gallstone fragments are trapped in the bile duct, the balloon may urge the stone fragments into the duodenum so that the stone fragments may pass out of the body naturally.

In a first embodiment of the invention, the catheter has proximal and distal regions, and a working lumen disposed between the proximal and distal regions. A balloon is coupled to an exterior surface of the catheter and configured to be inflated by an inflation lumen. At least one side port is disposed in a lateral surface of the catheter, the side port being in fluid communication with the working lumen of the catheter. The side port may be used to deliver a contrast medium.

The extraction basket has a contracted state in which it is adapted to be advanced longitudinally within the working lumen of the catheter, and further has an expanded state wherein the extraction basket is configured to capture and crush at least one relatively large stone. The extraction basket may comprise a plurality of resilient members. Each resilient member may comprise a proximal end coupled to the distal end of a control member, and further may comprise a distal end coupled to an atraumatic tip.

In a first method of operation, the catheter is inserted through a working channel of an endoscope. The balloon is provided in a deflated state, and the extraction basket is provided within the working lumen of the catheter in the contracted state. If a relatively large stone is detected, the extraction basket is distally advanced with respect to the catheter to deploy the plurality of resilient members. The plurality of resilient members may then be maneuvered to engage the relatively large stone.

In one embodiment, the extraction basket is configured to perform a mechanical lithotripsy procedure on the stone by using the plurality of resilient members to crush the stone into a plurality of smaller stone fragments. The mechanical lithotripsy procedure may be performed by retracting the plurality of resilient members proximally against a distal end of the catheter. If relatively large pieces are still present after the lithotripsy procedure, those pieces may be captured and removed using the extraction basket.

After the relatively large stones are removed, the extraction basket may be retracted proximally into the working lumen of the catheter to cause the extraction basket to assume the contracted state. The balloon of the catheter then is inflated and used to engage at least one of the relatively small stone fragments to urge the stone fragments and/or any sludge out of the body passage. Therefore, using the apparatus and methods described above, a comprehensive system is provided for capturing and removing relatively large stones, crushing relatively large stones into smaller stone fragments, and removing the smaller stone fragments and sludge.

Other systems, methods, features and advantages of the invention will be, or will become, apparent to one with skill in the art upon examination of the following figures and detailed description. It is intended that all such additional systems, methods, features and advantages be within the scope of the invention, and be encompassed by the following claims.

### BRIEF DESCRIPTION OF THE DRAWINGS

The invention can be better understood with reference to the following drawings and description. The components in the figures are not necessarily to scale, emphasis instead being placed upon illustrating the principles of the invention. Moreover, in the figures, like referenced numerals designate corresponding parts throughout the different views.

FIG. 1 is a side view of a first embodiment of a system in accordance with the present invention.

FIG. 2 is a side-sectional view showing the system of FIG. 1 having an extraction basket in a contracted state.

FIG. 3 is a cross-sectional view taken along line A-A of FIG. 2

FIG. 4 is a side view illustrating the system of FIGS. 1-3 having the extraction basket in an expanded state.

FIGS. 5A-5B are, respectively, perspective views of the distal ends of exemplary side-viewing and end-viewing endoscopes that may be used in conjunction with the system of FIGS. 1-4.

FIGS. 6A-6I illustrate a method for removing a stone or multiple stone fragments from a body passage using the system of FIGS. 1-4.

FIG. 7 is a side-sectional view of an alternative embodiment of the system of FIG. 1 having an extraction basket in a contracted state.

### DETAILED DESCRIPTION OF THE PREFERRED EMBODIMENTS

In the present application, the term “proximal” refers to a direction that is generally towards a physician during a medical procedure, while the term “distal” refers to a direction that is generally towards a target site within a patent's anatomy during a medical procedure. Thus, “proximal” and “distal” directions, relative to the bodily passageway in which the procedure is being performed, depend on the point of entry for the procedure (e.g., percutaneously or endoscopically).

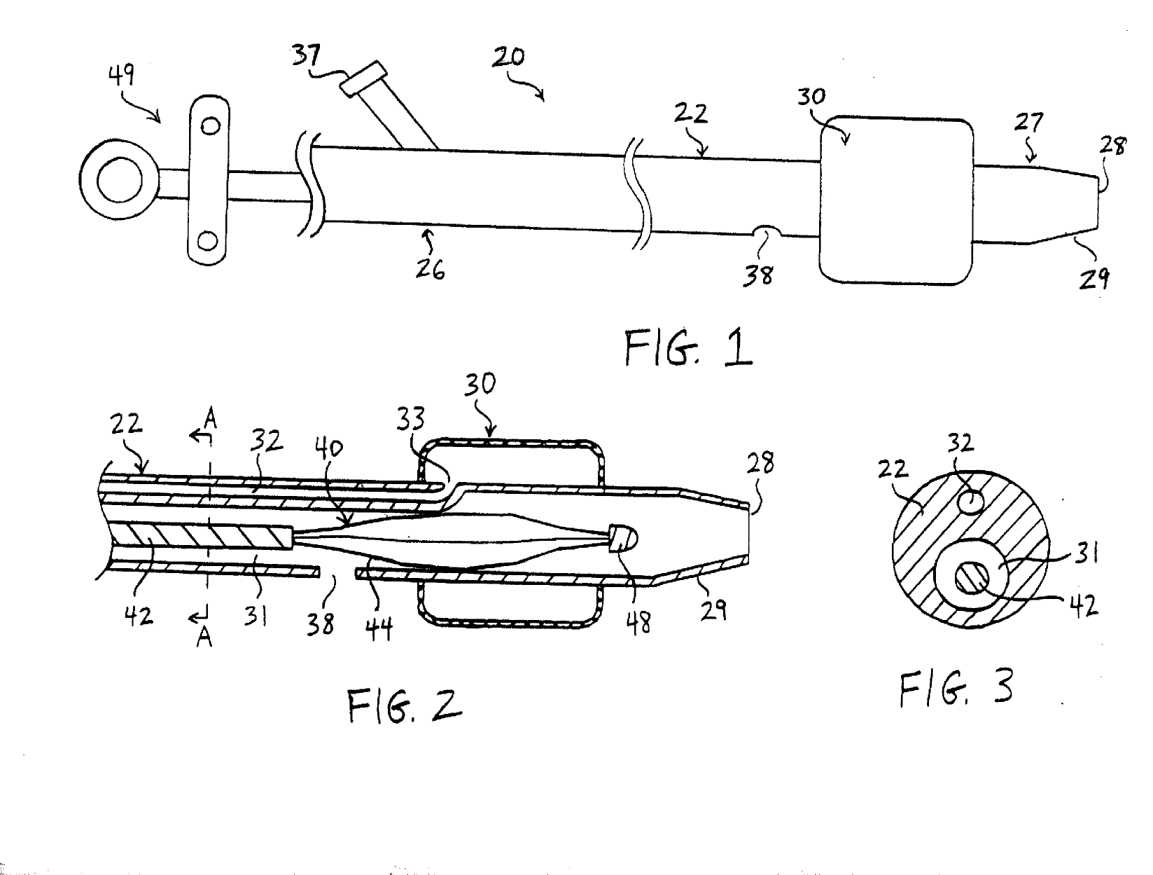

Referring now to FIG. 1, a first embodiment of a system according to the present invention is described Stone removal system 20 comprises catheter 22 having proximal and distal regions 26 and 27, respectively, and balloon 30 disposed on an exterior surface of distal region 27. Catheter 22 further comprises working lumen 31 and inflation lumen 32, as depicted in FIGS. 2-3.

System 20 further comprises extraction basket 40, which is disposed for longitudinal movement within working lumen 31 of catheter 22, as depicted in FIGS. 2-4. Extraction basket 40 is configured to entrap, and preferably crush, stones such as kidney stones and gallstones, as described in greater detail below.

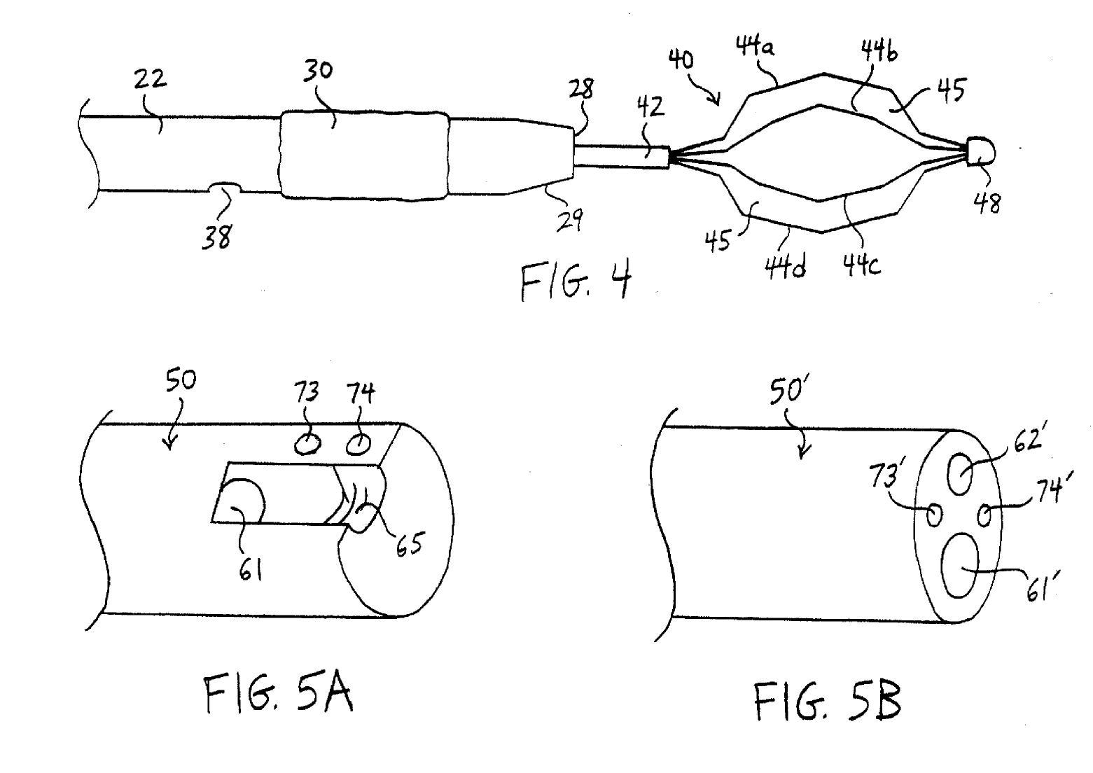

Extraction basket 40 preferably comprises at least three resilient members having contracted and expanded states. In the embodiments depicted herein, extraction basket 40 comprises four resilient members 44a-44d, as shown in FIG. 4, although greater or fewer resilient members may be employed. Moreover, the configuration of the resilient members, depicted in FIG. 4, is exemplary and alternative configurations may be used.

Each resilient member 44a-44d has a proximal end and a distal end. The proximal ends are each connected to control member 42, while the distal ends are each connected to atraumatic tip 48, as shown in FIG. 4. Control member 42 comprises proximal and distal ends. The proximal end of control member 42 may be coupled to handle 49, which is adapted to be manipulated in longitudinal and circumferential directions by a physician. The longitudinal and circumferential movement is imparted to the distal end of control member 42, which in turn is imparted to resilient members 44a-44d.

In the contracted state, resilient members 44a-44d are disposed substantially adjacent to one another and are constrained within the inner confines of working lumen 31 of catheter 22, as depicted in FIG. 2. By contrast, in the expanded state, resilient members 44a-44d may be advanced distally beyond the confines of working lumen 31 to assume an expanded configuration in which they bow radially outward between control member 42 and atraumatic tip 48, as depicted in FIG. 4. Resilient members 44a-44d may comprise stainless steel, a shape memory material such as nitinol, or another material adapted to assume the expanded state. In the expanded state, a plurality of open spaces 45 are formed between the resilient members are configured to allow stones to pass therethrough and into the interior volume of the basket, thereby allowing the stones to be entrapped, as described in further detail below.

Referring back to FIGS. 1-3, inflation lumen 32 of catheter 22 has a proximal end coupled to inflation port 37 and a distal end that terminates at side port 33, as shown in FIG. 2. Side port 33 is positioned within the inner confines of balloon 30, thereby placing inflation port 37 in fluid communication with balloon 30 and allowing inflation of the balloon.

Working lumen 31 spans from proximal region 26 to distal region 27. Since inflation lumen 32 preferably terminates beneath a proximal portion of balloon 30, the is diameter of working lumen 31 may be greater near distal end 28 of catheter 22, as shown in FIG. 2. Distal region 27 may comprise one or more tapered regions 29, which may facilitate entry into a bodily passageway, as discussed in greater detail below.

At least one side port 38 may be formed through a lateral surface of catheter 22 and placed in fluid communication with working lumen 31, as shown in FIG. 2. As will be explained in greater detail below, a contrast agent may be delivered through side port 38, via working lumen 31, to facilitate imaging of a bodily passageway during a stone removal procedure. Alternatively, side port 38 may be placed in fluid communication with an additional, separate lumen of catheter 22, e.g., to deliver a contrast agent through side port 38.

Catheter 22 may comprise a flexible, tubular member that may be formed from one or more semi-rigid polymers. For example, the catheter may be manufactured from polyurethane, polyethylene, tetrafluoroethylene, polytetrafluoroethylene, fluorinated ethylene propylene, nylon, PEBAX or the like.

Balloon 30 may be attached to distal region 27 of catheter 22 using any suitable adhesive, such as biocompatible glue, or alternatively, using heat-shrink tubing, heat bonding, laser bonding, welding, solvent bonding, one or more tie-down bands, or the like. Balloon 30 may be manufactured from a material such as Latex, Polyurethane, PEBAX, nylon, Hytrel, Arnitel, or other polymers that are suitable for use during an interventional procedure.

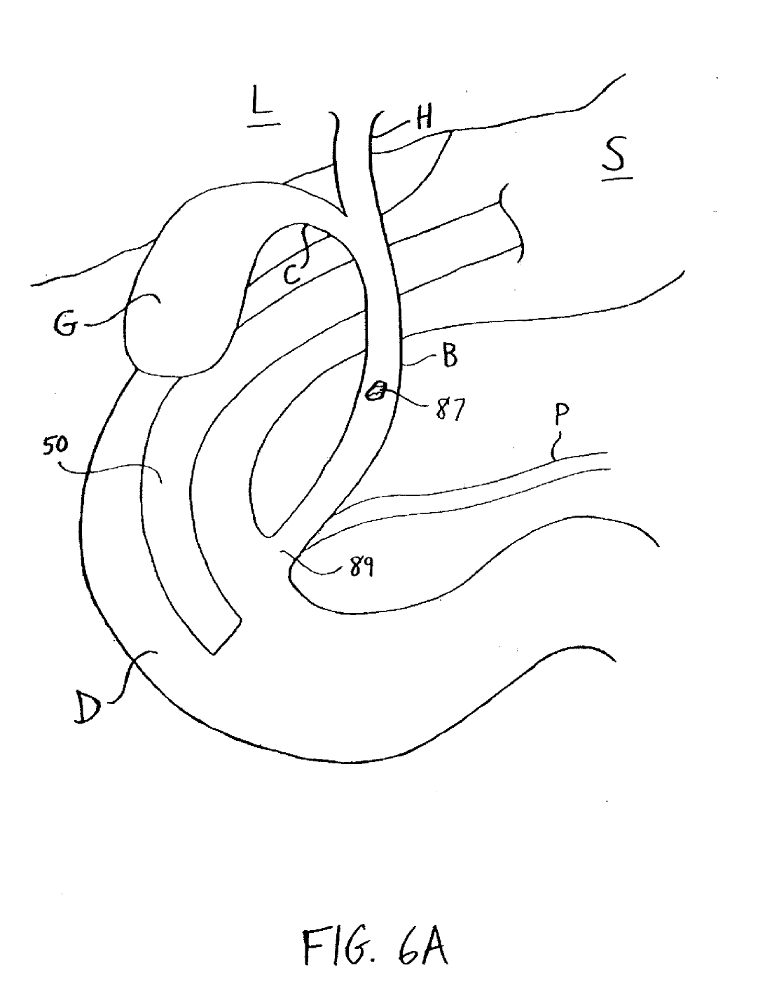

Referring now to FIGS. 5A-5B, exemplary endoscopes, which may be used in conjunction with system 20 during a stone removal procedure, are described. In FIG. 5A, side-viewing endoscope 50 comprises a conventional endoscope having proximal and distal ends. The distal end of endoscope 50 may comprise optical elements 73 and 74, which employ fiber optic components for illuminating and capturing an image to the side of, or distal to, the endoscope. Further, endoscope 50 preferably comprises working channel 61, which is sized to accommodate catheter 22 therein for purposes of longitudinally advancing the catheter to a target site. Guiding channel 65 may be formed near the distal surface of endoscope 50 to cause components advanced through working channel 61 to exit at a predetermined angle with respect to a longitudinal axis of endoscope 50. It will be apparent that while one working channel 61 is shown, endoscope 50 may comprise at least one more additional lumen or channel, such as an auxiliary lumen.

In FIG. 5B, an end-viewing endoscope 50′, which alternatively may be used in conjunction with system 20 during a stone removal procedure, is shown. Endoscope 50′ is similar to endoscope 50, with the main exception that optical elements 73′ and 74, are disposed on the distal end surface of endoscope 50′. Further, working channel 61′ extends through the distal end surface of endoscope 50′, as shown in FIG. 5B. Working channel 61′ also is sized to accommodate catheter 22 therein for purposes of longitudinally advancing the catheter to a target site. As shown, one auxiliary lumen 62′ is provided, although greater or fewer lumens/channels may be employed.

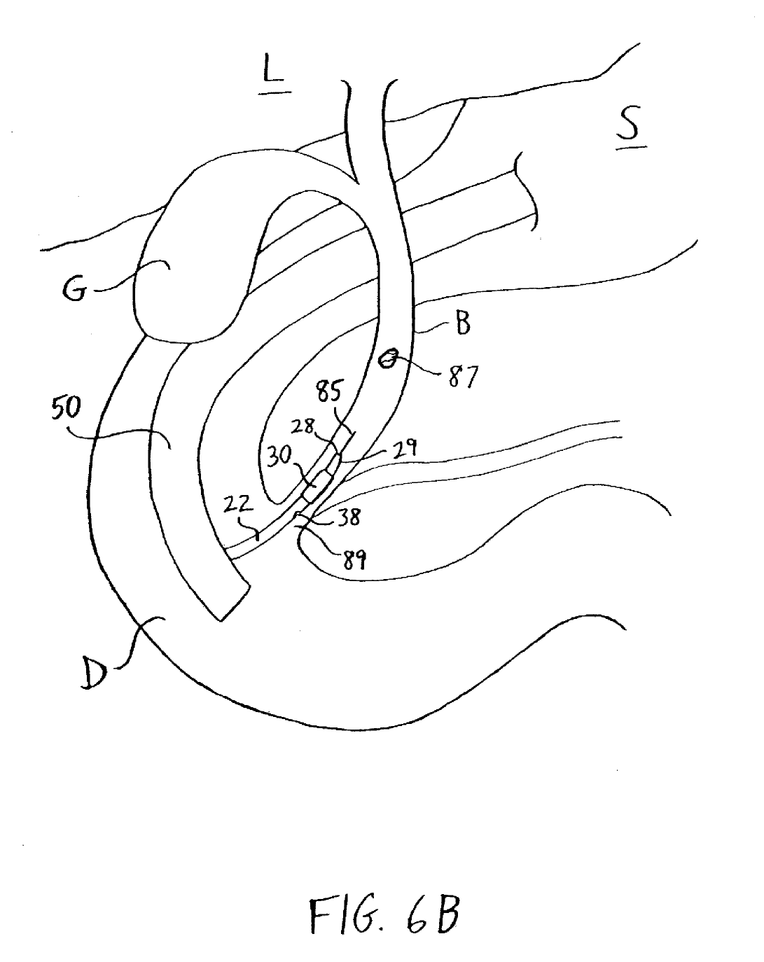

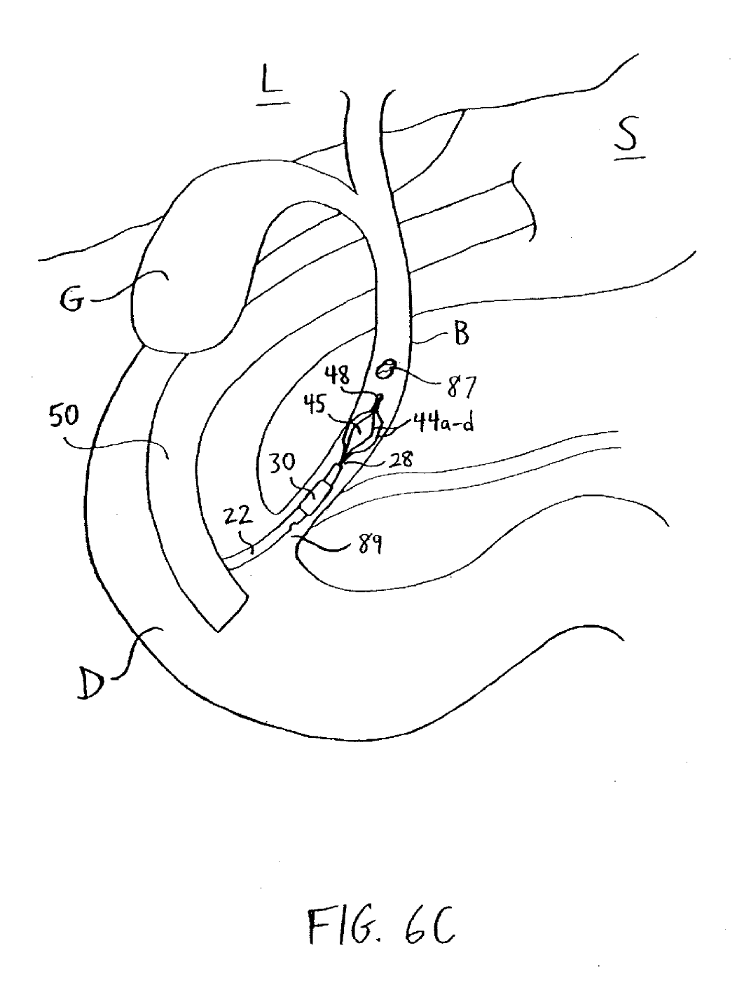

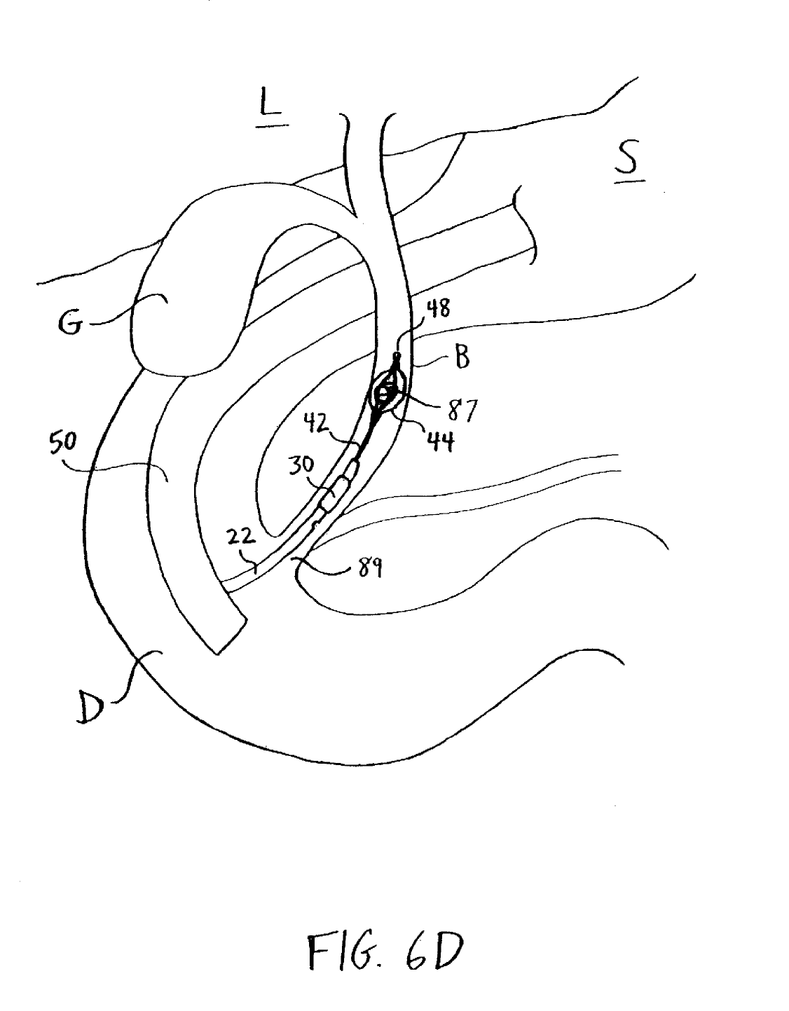

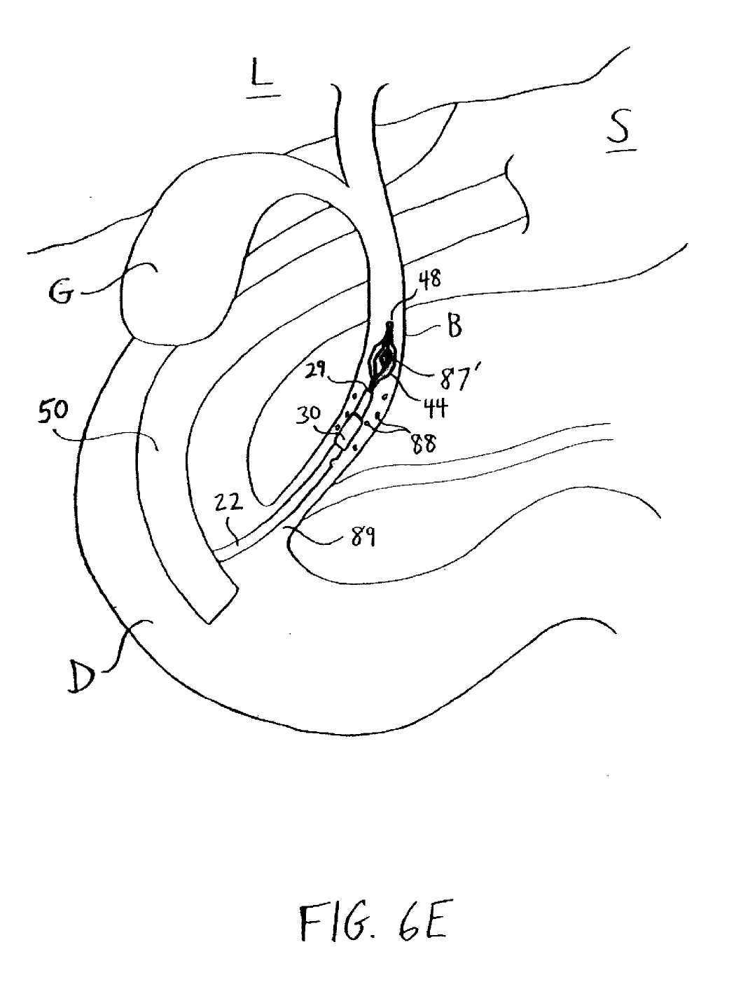

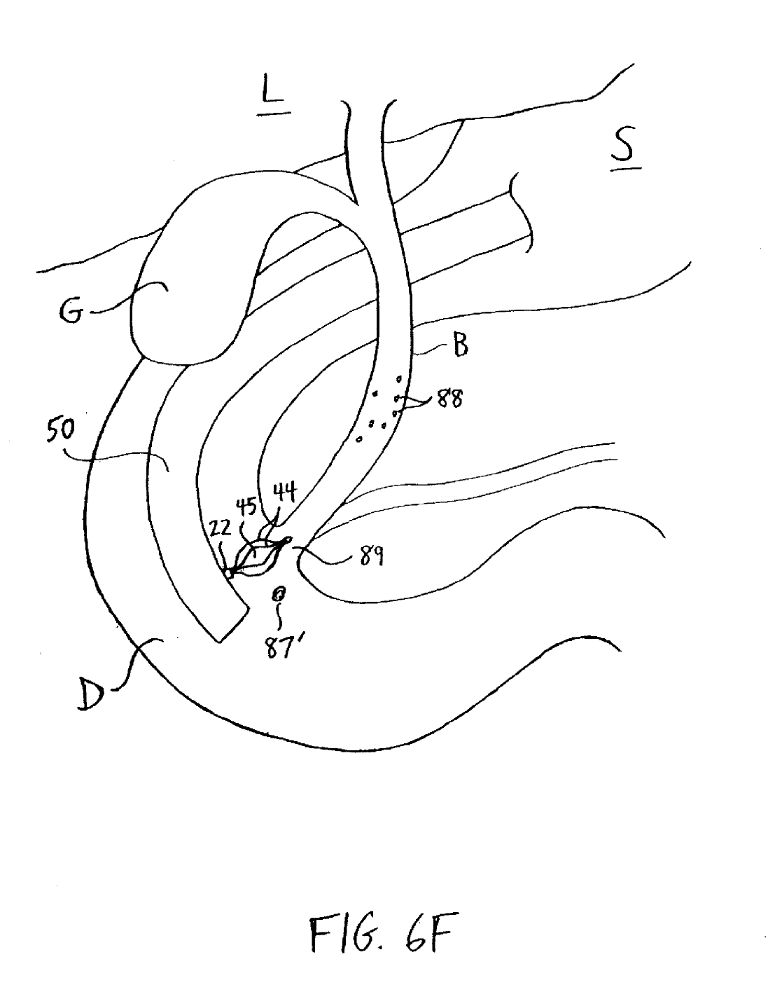

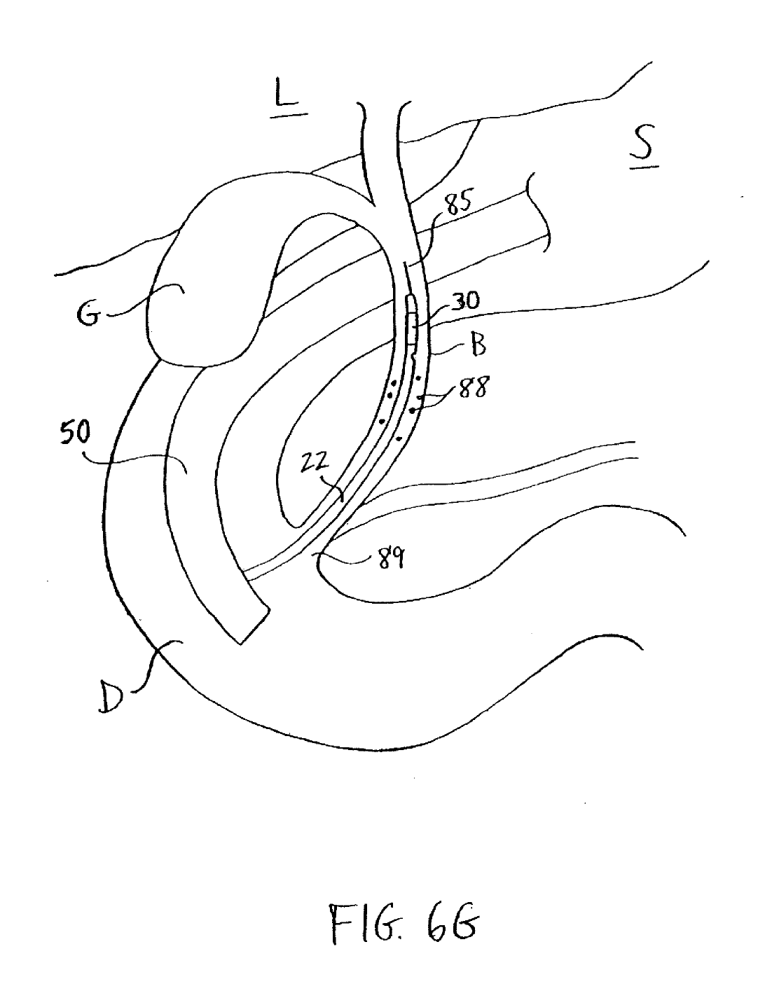

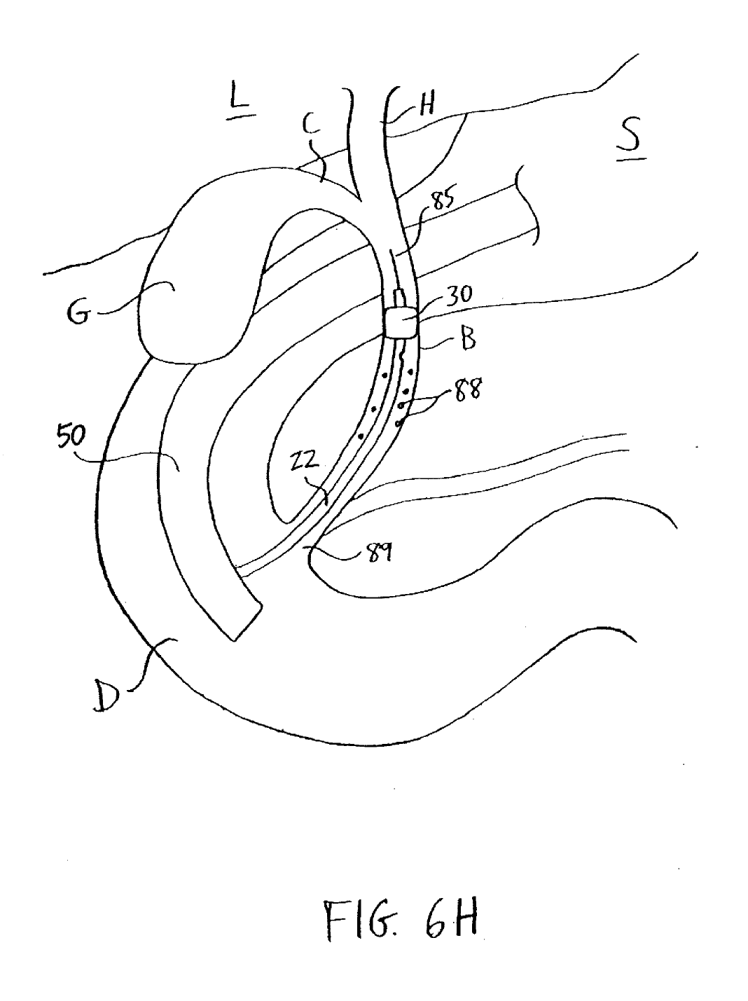

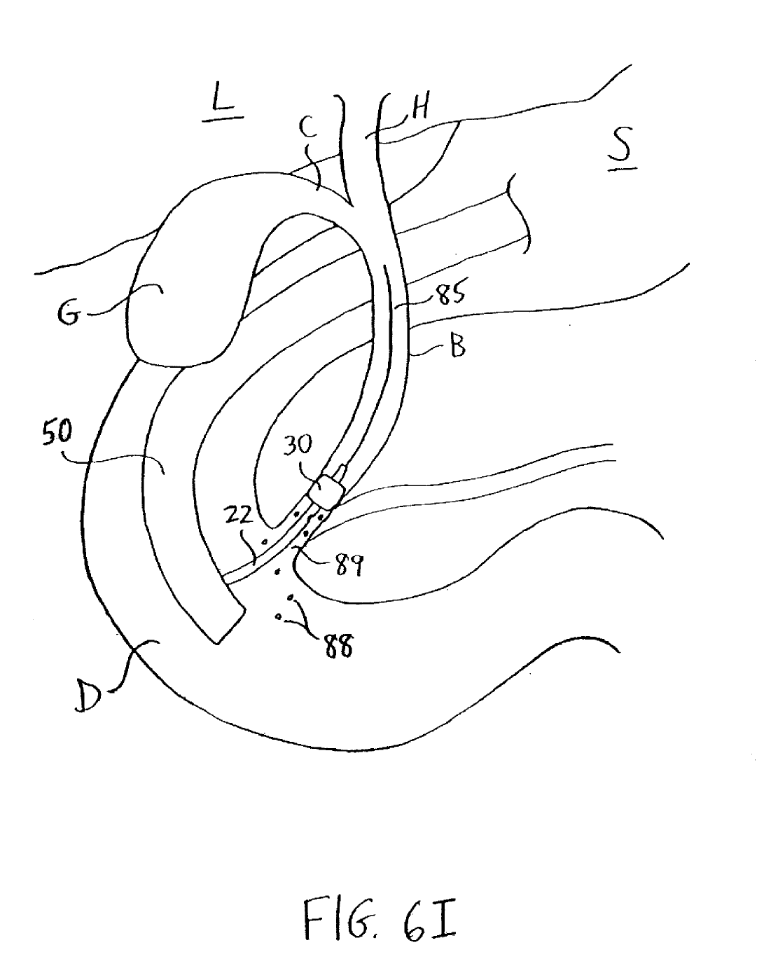

Referring now to FIGS. 6A-6I, an exemplary method for removing calculi, and in particular a gallstone, is described. As shown in FIG. 6A, the pertinent anatomy depicts cystic duct C leading from the gallbladder G into bile duct B. Hepatic duct H leads from liver L into bile duct B. The junction of cystic duct C and hepatic duct H form bile duct B, which extends towards sphincter of Oddi 89 and into duodenum D. Stomach S also empties into duodenum D, as shown in FIG. 6A. In this example, gallstone 87 has migrated from gallbladder G and has lodged within bile duct B. The gallstone may be trapped within bile duct B by the muscle of sphincter of Oddi 89.

In order to access bile duct B, an endoscopic retrograde cholangiopancreatography (ERCP) procedure may be performed. In a first step, a physician inserts endoscope 50 into a patient's mouth, through the esophagus, through stomach S, and into duodenum D, as schematically shown in FIG. 6A. In a preferred embodiment, endoscope 50 is a side-viewing endoscope, as described in FIG. 5A above. Alternatively, end-viewing endoscope 50′ of FIG. 5B may be employed. The distal end of endoscope 50 is positioned in the vicinity of sphincter of Oddi 89 and adjacent the papilla of Vater. The papilla of Vater may be located by visualizing the pancreas, and then tracing bile duct B and/or pancreatic duct P to the wall of duodenum D and the papilla of Vater.

Referring now to FIG. 6B, in a next step, catheter 22 is advanced through working channel 61 of endoscope 50 with balloon 30 in the deflated state (as shown in FIG. 4). At this time, extraction basket is disposed entirely within working lumen 31 of catheter 22, such that resilient members 44a-44d are in the contracted state (as depicted in FIG. 2). With balloon 30 deflated and resilient members 44a-44d contracted inside working lumen 31, catheter 22 is sized to be longitudinally advanced through working channel 61 of endoscope 50.

Working channel 61 of endoscope 50 may have an inner diameter of about 2.8-5.5 mm, while the overall diameter of endoscope 50 may be about 10-14 mm. Where the inner diameter of working channel 61 of endoscope 50 is about 5.5 mm, catheter 22 may comprise an outer diameter of about 4.0 mm and a working lumen 31 of about 3.0 mm in diameter to permit the passage of control member 42, resilient members 44a-44d and atraumatic tip 48 therein. These exemplary dimensions are used for reference purposes and are not intended to be limiting.

If necessary, a sphincterotomy may be performed at sphincter of Oddi 89 to facilitate access into bile duct B using techniques that are known in the art. The sphincterotomy may be performed using an auxiliary lumen of endoscope 50, Optionally, wire guide 85 may be advanced out of endoscope 50, through sphincter of Oddi 89, and into bile duct B. Wire guide 85 may be inserted through working lumen 31 of catheter 22 alongside extraction basket 40, or alternatively, through a separate and dedicated wire guide lumen (not shown) furnished within catheter 22. Distal end 28 of catheter 22 is then advanced over wire guide 85 and disposed proximal (downstream) of gallstone 87, as depicted in FIG. 6B. Distal end 28 of catheter 22 preferably comprises one or more tapered regions 29 to facilitate advancement through sphincter of Oddi 89.

In order to facilitate suitable imaging of bile duct B, radioscopy, fluoroscopy, or the like may be performed. In one example, a contrast medium may be delivered though working lumen 31, such that the contrast medium flows around extraction basket 40 and exits through side port 38. The contrast medium is injected into bile duct B. In another example, catheter shaft 22 may comprise one or more radiopaque bands to ascertain its position within bile duct B. Alternatively, if a stone is located in the vicinity of sphincter of Oddi 89, and the sphincter is sufficiently dilated, then the catheter, stone and other items may be viewed directly using endoscope 50.

Referring now to FIG. 6C, in a next step, control member 42 is advanced distally with respect to catheter 22, thereby causing atraumatic tip 48 and resilient members 44a-44d to be advanced beyond distal end 28 of catheter 22. At this time, resilient members 44a-44d are no longer constrained and, since they are biased, they assume their radially expanded state, as depicted in FIG. 6C. Although extraction basket 40 is depicted as being deployed proximal (downstream) of gallstone 87, the basket also may be deployed distal (upsteam) or adjacent to gallstone 87, depending on the nature and location of the gallstone.

Referring to FIG. 6D, with resilient members 44a-44d of the extraction basket deployed in the vicinity of gallstone 87, control member 42 may be manipulated to capture gallstone 87 between resilient members 44a-44d. In particular, control member 42 may be advanced proximally and distally, and rotated circumferentially, to allow gallstone 87 to enter through open spaces 45 formed between resilient members 44a-44d (see FIG. 4), so as to entrap the stone between the resilient members.

Referring now to FIG. 6E, in a next step, control member 42 is retracted proximally with respect to catheter 22 so that resilient members 44a-44d may engage distal end 28 of catheter 22. As resilient members 44a-44d engage distal end 28, the resilient members collapse around gallstone 87, which is trapped therein. As resilient members 44a-44d are further retracted proximally, a compressive force is applied to gallstone 87, which crushes the stone into smaller gallstone piece 87′ and smaller stone fragments 88, as depicted in FIG. 6E. Gallstone piece 87′ is smaller than gallstone 87, but larger than stone fragments 88, such that it may remain captured between resilient members 44a-44d.

In an alternative embodiment, a different extraction basket may be provided in which one or more cables (not shown) are coupled to one or more resilient members 44a-44d, thereby enabling independent proximal retraction of the one or more resilient members 44a-44d with respect to one another to facilitate capture and/or crushing of gallstone 87.

Resilient members 44a-44d may be reinforced to ensure that they have sufficient strength to overcome the resistive force provided by gallstone 87, i.e., so that the resilient members do not rupture. Moreover, distal end 28 of catheter 22 may be reinforced, e.g., using a stainless steel frame, to ensure that it has sufficient strength to overcome the force provided by the retraction of resilient members 44a-44d, i.e., thereby reducing the likelihood of the catheter end kinking or bending.

In an alternative method step, a shock wave lithotripsy probe (not shown) may be used in conjunction with system 20 to crush gallstone 87. For example, the lithotripsy probe may be inserted through an auxiliary lumen (not shown) of catheter 22 until the probe exits distal to the catheter. The probe may be advanced towards gallstone 87 while resilient members 44a-44d hold the gallstone securely in place. With the gallstone held steady, shock waves may be generated, for example, using either electrohydraulic or laser technology. In an electrohydraulic lithotripsy procedure, a vaporizing fluid is delivered in the vicinity of gallstone 87 and voltage is applied to electrodes located at the distal end of the probe to produce shock waves at the surface of gallstone 87. If this technique is employed, the vaporizing fluid may be delivered through working lumen 31 in an annular space around control member 42 (see, e.g., FIG. 3). Alternatively, the vaporizing fluid may be delivered through the auxiliary lumen of the catheter that houses the lithotripsy probe.

In a laser lithotripsy procedure, light is converted into thermal energy at the surface of gallstone 87. Various commercial electrohydraulic and laser lithotripsy systems are currently available for performing endoscopic lithotripsy.

Therefore, the use of mechanical lithotripsy by retracting resilient members 44a-44d against catheter 22, or the use of electrohydraulic or laser lithotripsy, may form a reduced size gallstone piece 87′ and smaller stone fragments 88, as depicted in FIG. 6E. Gallstone piece 87′ may be captured between resilient members 44a-44d. Stone fragments 88, on the other hand, are so small that they may not be captured by extraction basket 40, as they will escape between adjacent resilient members 44a-44d.

Referring now to FIG. 6F, with resilient members 44a-44d held securely against distal end 28 of catheter 22, control member 42 and catheter 22 may be simultaneously retracted in a proximal direction through sphincter of Oddi 89. Resilient members 44a-44d may apply a compressive force upon gallstone piece 87′ during the retraction to secure gallstone piece 87′ within the basket. Once resilient members 44a-44d are positioned within duodenum D, control member 42 may be manipulated by being advanced proximally or distally, or by being rotated circumferentially, to allow gallstone piece 87′ to escape the basket through open spaces 45, as depicted in FIG. 6F. Gallstone piece 87′ is released into duodenum D and may pass naturally through the body.

Extraction basket 40 then may be fully retracted into the confines of working lumen 31 by proximally retracting control member 42 while holding catheter 22 steady. This causes resilient members 44a-44d to collapse, as generally shown in FIG. 2. Catheter 22 then may be advanced distally back into bile duct B, for example, over wire guide 85, as shown in FIG. 6G.

If a physician deems that additional gallstone pieces are disposed within bile duct B that are large enough to be captured within extraction basket 40, then some or all of the steps discussed in FIGS. 6C-6F may be repeated to deploy extraction basket 40, capture the stone within extraction basket 40, crush the stone, and remove the stone into the duodenum via the basket. Assuming, however, that smaller stone fragments 88 cannot be captured within extraction basket 40, then catheter 22 is advanced distally within bile duct B until it has passed some or all stone fragments 88, as shown in FIG. 6G.

Referring now to FIGS. 6H-6I, in a next step balloon 30 is inflated via the provision of fluid to inflation port 37 and inflation lumen 32. The balloon is inflated to engage an interior wall of bile duct B at a location distal to stone fragments 88, as shown in FIG. 6H. The physician may then retract catheter 22 proximally to cause balloon 30 to urge stone fragments 88 towards sphincter of Oddi 89, as shown in FIG. 6I. Balloon 30 may also be utilized to facilitate dilation of sphincter of Oddi 89 so as to permit stone fragments 88 to pass more freely into duodenum D. Once the stone fragments are in duodenum D, they will pass naturally through the patient via the intestinal pathway.

It will be apparent that while mechanical and intraductal shock wave lithotripsy has been described, other lithotripsy techniques may be used. For example, extracorporeal shock wave lithotripsy may be used to disintegrate a large stone, prior to the introduction of system 20 into bile duct B.

Advantageously, a comprehensive system is provided for capturing and removing relatively large stones, crushing relatively large stones into smaller stone fragments, and removing the smaller stone fragments and sludge, without the need to insert and remove various devices through an endoscope during the procedure. Since there is no need to remove one device prior to the introduction and advancement of a subsequent device, the overall operation time during a stone removal procedure may be reduced.

It will be apparent that while FIGS. 6A-6I depict a method for removing gallstones in bile duct B using an ERCP procedure, bile duct B alternatively may be accessed laparoscopically using techniques that are known in the art. The laparoscopic access of bile duct B, to facilitate removal of a gallstone, is described in greater detail in commonly-assigned U.S. Provisional patent application Ser. No. 11/747,570 (attorney docket 10000/899), which is hereby incorporated by reference in its entirety. Specifically, after an appropriate abdominal incision, a small incision may be made in cystic duct C. Wire guide 85 then is inserted through the incision and fluoroscopically advanced through cystic duct C and into bile duct B. Catheter 22 may then be inserted over wire guide 85, through cystic duct C and into bile duct B. If gallstone 87 is relatively large, extraction basket 40 may be deployed to capture the stone, crush the stone, and/or remove the stone into duodenum D via the basket, as generally described above. If gallstone 87 is relatively small, such that lithotripsy is not needed or the stone cannot be captured by extraction basket 40, then wire guide 85 may be advanced distally (downstream) past gallstone 87 and through bile duct B, though sphincter of Oddi 89 and into duodenum D. Using this laparoscopic technique, balloon 30 then may be inflated and advanced distally (downstream) over wire guide 85 towards duodenum D. The distal advancement of catheter 22 within bile duct B urges stone fragments 88 towards and through sphincter of Oddi 89. Balloon 30 may also be utilized to facilitate dilation of sphincter of Oddi 89 so as to permit stone fragments 88 to pass therethrough and into duodenum D. Once the stone fragments are in duodenum D, they will pass naturally through the patient via the intestinal pathway.

Finally, it will be apparent that while the above embodiments have described system 20 that may be used to treat gallstones that have migrated into the bile duct, the apparatus and methods may be used to remove calculi or other particulate matter in other anatomical passages, such as kidney stones in the ureter or kidneys, and so forth.

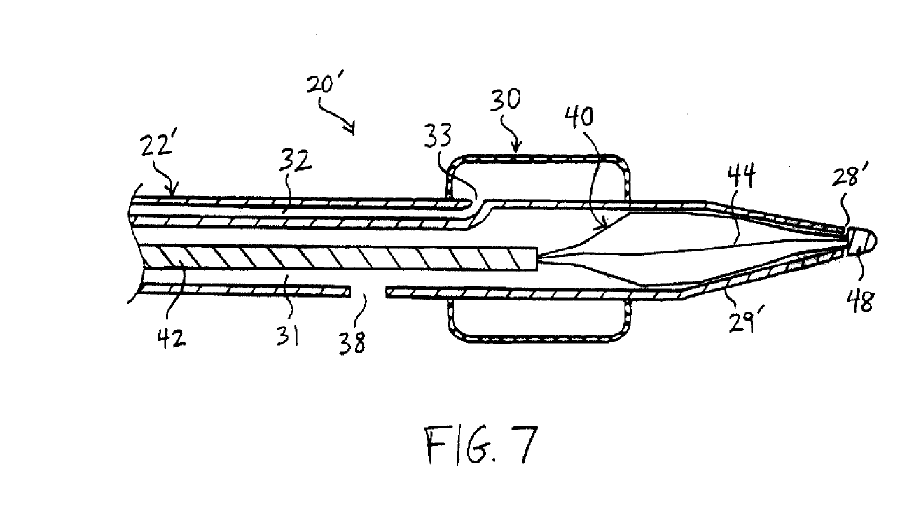

Referring now to FIG. 7, in an alternative embodiment, stone removal system 20′ is similar to stone removal system 20 of FIGS. 1-6, with a main exception that tapered region 29′ of catheter 22′ is slightly longer and configured to cooperate with atraumatic tip 48. Preferably, distal end 28′ of catheter 22′ is substantially flush with an outer diameter of atraumatic tip 48 when extraction basket 40 is in the contracted state within working lumen 31, as depicted in FIG. 7. Accordingly, if a contrast agent is delivered through working lumen 31, the contrast agent will be urged through side port 38 only, and will not flow through distal end 28′ due to the positioning of the atraumatic tip 48. The operation of stone removal system 20′ is substantially identical to the method steps described for stone removal system 20 in FIGS. 6A-6I above.

While various embodiments of the invention have been described, it will be apparent to those of ordinary skill in the art that many more embodiments and implementations are possible within the scope of the invention. Accordingly, the invention is not to be restricted except in light of the attached claims and their equivalents.

We claim:

1. Apparatus suitable for removing at least one stone in a body passage, the apparatus comprising:
a catheter having proximal and distal regions, and a working lumen disposed between the proximal and distal regions;
a balloon coupled to an exterior surface of the catheter, the balloon having deflated and inflated states;
an inflation lumen disposed between the proximal and distal regions of the catheter, wherein the inflation lumen is in fluid communication with an interior of the balloon to inflate the balloon;
an extraction basket coupled to a control member, the extraction basket comprising a plurality of resilient members having a contracted state suitable for advancement within the working lumen of the catheter and an expanded state configured to capture at least one stone; and
a proximal assembly comprising a handle and an inflation port, wherein the handle is operably coupled to the control member of the extraction basket and wherein the inflation port is in fluid communication with the inflation lumen,
wherein a distal end of the catheter is configured to engage the resilient members of the extraction basket to allow a crushing force to be applied to the stone as the resilient members are retracted in a proximal direction against the distal end of the catheter, and
wherein the balloon is configured to urge at least one smaller stone fragment in a direction out of the body passage when in the inflated state.

2. The apparatus of claim 1 wherein the distal end of the catheter is reinforced using a stainless steel frame that has a sufficient strength to reduce bending thereof caused by retraction of the plurality of resilient members.

3. The apparatus of claim 1 wherein each of the resilient members comprises a proximal end coupled to a distal end of the control member, and wherein each of the resilient members further comprises a distal end coupled to an atraumatic tip.

4. The apparatus of claim 3 wherein the atraumatic tip is disposed distal to the catheter, and wherein the atraumatic tip comprises a proximal portion having an outer diameter that is substantially flush with an outer diameter at the distal end of the catheter.

5. The apparatus of claim 4 wherein the distal region of the catheter comprises a taper having a reduced outer diameter, and wherein the outer diameter of the atruamatic tip is substantially flush with the reduced outer diameter of the catheter.

6. The apparatus of claim 1 wherein the extraction basket is configured to perform a mechanical lithotripsy procedure on a relatively large stone to crush the stone into the one or more smaller stone fragments.

7. The apparatus of claim 1 wherein the catheter is adapted for longitudinal movement through a working channel of an endoscope.

8. The apparatus of claim 1 further comprising at least one side port disposed in a lateral surface of the catheter, the side port being in fluid communication with the working lumen of the catheter.

9. A method suitable for removing at least one stone in a body passage, the method comprising:
providing a catheter having proximal and distal regions, and a balloon coupled to an exterior surface of the catheter, wherein the balloon is provided in a deflated state;
providing an extraction basket adapted to be advanced longitudinally within a working lumen of the catheter, wherein the extraction basket is provided in a contracted state;
distally advancing the catheter and the extraction basket substantially simultaneously into the body passage and in proximity to a stone;
distally advancing the extraction basket with respect to the catheter to deploy a plurality of resilient members of the extraction basket;
capturing the stone between the plurality of resilient members;
performing a mechanical lithotripsy procedure on the stone, using the plurality of resilient members, to crush the stone into a plurality of stone fragments;
inflating the balloon;
engaging at least one of the stone fragments with the inflated balloon; and
urging at least one of the stone fragments out of the body passage using the inflated balloon.

10. The method of claim 9 wherein performing a mechanical lithotripsy procedure comprises retracting the plurality of resilient members proximally against a distal end of the catheter.

11. The method of claim 9 further comprising substantially simultaneously retracting the catheter and the extraction basket to remove a stone, captured between the plurality of resilient members, from the body passage.

12. The method of claim 9 further comprising proximally retracting the extraction basket into the working lumen of the catheter to cause the extraction basket to assume the contracted state, prior to the step of engaging stone fragments with the inflated balloon.

13. The method of claim 9 further comprising:
coupling proximal ends of the plurality of resilient members to a distal end of a control member;
coupling distal ends of the plurality of resilient members to an atraumatic tip; and
longitudinally advancing the control member to effect longitudinal movement of the plurality of resilient members.

14. The method of claim 13 further comprising circumferentially rotating the control member to effect circumferential rotation of the plurality of resilient members.

15. The method of claim 9 further comprising:
capturing a stone lodged in a bile duct between the plurality of resilient members;
simultaneously retracting the catheter and the plurality of resilient members to retract the stone into the duodenum; and
manipulating the plurality of resilient members to release the stone into the duodenum.

16. A method suitable for removing at least one stone in a body passage, the method comprising:
providing a catheter having proximal and distal regions, and a balloon coupled to an exterior surface of the catheter, wherein the balloon is provided in a deflated state;
providing an extraction basket adapted to be advanced longitudinally within a working lumen of the catheter, wherein the extraction basket is provided in a contracted state;
distally advancing the catheter and the extraction basket substantially simultaneously into the body passage and in proximity to a stone;
distally advancing the extraction basket with respect to the catheter to deploy a plurality of resilient members;
manipulating the plurality of resilient members to capture a relatively large stone;
removing the captured relatively large stone from the body passage;
proximally retracting the extraction basket into the working lumen of the catheter to cause the extraction basket to assume the contracted state;
inflating the balloon;
engaging at least one relatively small stone fragment with the inflated balloon; and
urging at least one of the relatively small stone fragments out of the body passage using the inflated balloon.

17. The method of claim 16 further comprising using the extraction basket to perform a mechanical lithotripsy procedure on the relatively large stone to crush the relatively large stone into smaller fragments.

18. The method of claim 17 further comprising retracting the extraction basket proximally against a distal end of the catheter to perform the mechanical lithotripsy procedure.

19. The method of claim 16 further comprising:
capturing the relatively large stone within a bile duct between the plurality of resilient members;
simultaneously retracting the catheter and the plurality of resilient members to retract the relatively large stone into the duodenum; and
manipulating the plurality of resilient members to release the relatively large stone into the duodenum.

20. The method of claim 16 further comprising:
providing at least one side port disposed in a lateral surface of the catheter, wherein the side port is placed in fluid communication with the working lumen of the catheter; and
injecting a contrast medium through the side port.
